(19) United States
(12) Patent Application Publication
(10) Pub. No.: US 2024/0237885 A1
(43) Pub. Date: Jul. 18, 2024

(54) Micro Optics and Fiber Endoscope

(71) Applicant: Printoptix GmbH, Stuttgart (DE)

(72) Inventors: Simon Thiele, Stuttgart (DE); Nils Fahrbach, Stuttgart (DE); Andrea Toulouse, Stuttgart (DE); Alois Herkommer, Aalen (DE)

(21) Appl. No.: 18/243,810

(22) Filed: Sep. 8, 2023

(30) Foreign Application Priority Data
Sep. 9, 2022 (DE) 10 2022 122 923.8

(51) Int. Cl. A61B 1/00; A61B 1/04

(52) U.S. Cl. CPC A61B 1/00167; A61B 1/00096; A61B 1/00179; A61B 1/044

(57) ABSTRACT

Micro-optics, in particular for connection to a fiber endoscope, including an entry face and an exit face, the exit face being connectable to an end face of the fiber endoscope, and dispersive optics arranged between the entry face and the exit face, so that light passing the dispersive optics is split at the exit face depending on the wavelength, the micro-optics being monolithic.

### CROSS-REFERENCE TO RELATED APPLICATION

This application claims priority to German Patent Application No. 10 2022 122 923.8 filed Sep. 9, 2022, the disclosure of which is hereby incorporated by reference in its entirety.

### BACKGROUND OF THE INVENTION

### Field of the Invention

The present invention relates to micro-optics in particular for use with a fiber endoscope. The present invention further relates to a method for manufacturing such micro-optics, as well as to a fiber endoscope comprising such micro-optics.

Endoscopy is nowadays a common method for surgical or therapeutic procedures in minimally invasive surgery. Here, it is necessary to obtain images of the surgical site with a high resolution and a good image quality. Therefore, efforts are made to increase the image resolution of such endoscopes and at the same time reduce imaging errors. In order to increase the flexibility of the endoscopes, it is known to use optical wave guides as a transmission medium, which can adapt flexibly to the physiological situation at the surgical site.

In this context, the measures for enhancing the image resolution are limited by the specific requirements of the application. For example, an optical system has to be provided that can be small. However, the miniaturization of such optical system usually results in a deterioration of the image quality and the image resolution. As a rule, image information is transmitted via the pixel of a digital image sensor or the fiber cores of a so-called image conducting fiber. Since a fixed minimum size exists for these pixels/fiber cores, the number of available pixels decreases in the case of a reduced endoscope diameter, and the image resolution decreases with the same.

### Description of Related Art

For example, it is known from U.S. Pat. No. 3,449,037 to design a fiber as an orderly bundle of optical waveguides and to use the same as a transmission medium with a special device for increasing the resolution. In this case, the system comprises a first optical arrangement with a dispersive element. The light is split by the dispersive element as a function of the wavelength. A pixel captured by the first optical element is thus imaged to different locations on the end facet or end face of the fiber and is thus transmitted through different optical waveguides. A second optical arrangement is provided at the second end of the fiber, which also comprises a dispersive element with an arrangement mirroring that of the first dispersive element, so that the pixel is recomposed from the individual color components transmitted by different optical waveguides of the fiber. This system can achieve an increased image resolution. However, the system is difficult to miniaturize. When the first optical arrangement is miniaturized, the degree of splitting, i.e. the distance between the individual color components of a pixel on the end facet of the fiber, is automatically reduced as well. A result of such miniaturization is that in such cases prisms with very large wedge angles are required for reasonable transverse splitting. The strongly inclined surfaces lead to substantial image errors in the system, which negatively affect both the image resolution and the distortion of the image. The use of diffraction gratings, which are much easier to miniaturize compared to prisms, usually results in undesired stray light and reduced image contrast.

As a consequence, a reasonable miniaturization of the system of U.S. Pat. No. 3,449,037 is not possible, so that the use of such a solution in an endoscope is not yet possible.

### SUMMARY OF THE INVENTION

It is an object of the present invention to provide micro-optics for a fiber endoscope which are particularly compact and increase the resolution of the fiber endoscope without a loss of image quality.

The object is achieved with micro-optics as described herein, a method for manufacturing such micro-optics as described herein, as well as an endoscope as described herein.

The micro-optics, in particular for connection to a fiber endoscope, has an entry face and an exit face. Here, the exit face can be connected to an end face of the fiber endoscope. The end face of the fiber endoscope can be an end facet of the fiber of the fiber endoscope, for example. In this case, light to be transmitted by the fiber endoscope, for example from the surgical site, enters the micro-optics via the entry face and is coupled into the fiber of the fiber endoscope at the exit face and is transmitted. In the process, pixels can be imaged or focused onto the exit face by the micro-optics. In particular, the entry face and the exit face are configured to be parallel. However, other configurations are also conceivable.

Further, according to the invention, the micro-optics comprises a dispersive optics arranged between the entry face and the exit face, wherein the dispersive optics is configured to split light, which passes the dispersive optics, at the exit face depending on the wavelength and to image the light onto the exit surface at different locations as a function of the wavelength. A pixel whose light enters the micro-optics at the entry face is thus split by the dispersive optics in dependence on the wavelength and imaged to the exit face, so that different wavelengths of the pixel are transmitted via different cores of the optical waveguide of the fiber endoscope.

According to the invention, the micro-optics are of monolithic design in this case. Due to the monolithic design of the micro-optics, it is possible to design the same particularly compact. Specifically, the orientation of the individual components of the micro-optics relative to each other is fixed due to the monolithic design. Thus, it is possible to miniaturize the micro-optics to a sufficient extent for use on an endoscope.

The micro-optics are preferably cylindrical.

Preferably, the micro-optics have a diameter of less than 1 mm. preferred less than 800 μm and particularly preferred less than 500 μm.

The dispersive optics preferably have a prism which is configured in particular as a direct-vision prism, a dispersion prism or an Amici prism. The prism provides a simply structured dispersive element of the dispersive optics for splitting the light into the micro-optics in dependence on the wavelength.

The dispersive optics preferably comprises a transmission grating. The transmission grating also provides a dispersive element for a wavelength-dependent splitting of the individual pixels, so that the individual pixels can be transmitted via the optical waveguides of the fiber of the fiber endoscope in dependence on the wavelength.

The transmission grating preferably comprises a constant periodicity. As an alternative, the transmission grating has a non-constant periodicity which is also referred to as chirp. As a result, the imaging properties of the transmission grating can be adapted in a suitable manner to influence the imaging properties. In particular, by controlling the periodicity, it is possible to achieve an almost linear relationship between the wavelength and the splitting. Thereby, an improvement in image quality can be achieved and at the same time a recombination of the individual pixels can be facilitated.

The exit face of the transmission grating is preferably arranged parallel to the exit face of the micro-optics. As an alternative, the exit surface of the transmission grating is not arranged parallel to the exit face of the micro-optics.

Preferably, the transmission grating is stair-shaped.

Preferably, the dispersive optics comprise more than one dispersive element. For example, the dispersive optics may comprise more than one transmission grating, more than one prism or a combination of one or more prisms and one or more transmission gratings.

The dispersive optics are preferably inclined with respect to the entry face and/or the exit face. Thereby, it is possible to suitably adapt the imaging property of the dispersive optics in order to avoid imaging errors. In classical optics of the prior art composed of individual components, such an inclination is difficult to achieve with high precision and usually requires manual readjustment.

The dispersive optics preferably comprise a first surface directed towards the entry face. As an alternative or in addition, the dispersive optics comprise a second surface directed towards the exit face. If the dispersive optics have both a first surface and a second surface, the second surface is arranged opposite the first surface. It may be that the dispersive optics have no first surface or no second surface, since the dispersive optics is combined with the entry face or the exit face, for example, so that the entry face can be understood as the first surface of the dispersive optics and the exit face can be understood as the second surface of the dispersive optics, respectively.

Preferably, the first surface is curved. As an alternative or in addition, the second surface is curved. By a curved design of the surface of the dispersive optics, the imaging properties of the dispersive optics can be selected precisely in order to thereby prevent imaging errors and to further increase the imaging quality. In classical optics of the prior art composed of individual components, such a curvature is difficult to achieve with high precision and usually requires manual readjustment of the individual components relative to each other.

Preferably, the first surface and/or the second surface are not rotationally symmetrical and are designed in particular as freeform surfaces. As a result, the freedom in designing the dispersive optics is further increased, so that possible occurring imaging errors can be compensated for to further increase the imaging quality of the micro-optics. In particular when using a prism as the dispersive optics, the dispersive optics has a non-rotationally symmetrical design. Due to the non-rotationally symmetrical shape of the surface, this can be compensated for in order to obtain the most error-free imaging possible across the entire entry face or exit face.

Preferably, the splitting of the light on the exit face takes place along an axis perpendicular to an axial direction of the micro-optics or the fiber endoscope. Here, the axial direction may substantially represent an axis perpendicular to the entry face and/or the exit face and for example coincide with the optical axis of one or more optical elements, such as e.g. the dispersive optics. Thus, the splitting by the dispersive optics takes place along an axis perpendicular to this axial direction of the micro-optics.

Preferably, the splitting is substantially linearly dependent on the wavelength. Thus, a doubling of the wavelength leads to a doubling of the splitting at the exit surface in particular along the axis perpendicular to the axial direction of the micro-optics. This allows for a simple recombination of the individual wavelengths of a pixel and non-linearities do not have to be compensated for in a complex manner during recombination.

The micro-optics preferably consists of a transparent material and in particular an acrylate or an epoxy. In this context, transparent refers to a property of the material to substantially transmit light in the near UV, the visible wavelength range and/or the near infrared. Here, substantially means that more than 50% of the light passes the material, preferably more than 70%, more preferred more than 90% and particularly preferred more than 95%.

Preferably, the micro-optics are produced by 3D laser writing and in particular by means of a two-photon lithography process. In 3D laser writing, a photoresist is cured by a focused laser beam to create the desired structure. Thus, small structures can be formed precisely and the desired miniaturization of the micro-optics can be achieved. At the same time, the method of 3D laser writing achieves a high reproducibility, so that micro-optics with the desired optical properties can be reliably produced with an enhanced imaging quality with a simultaneous increase in resolution.

The micro-optics preferably comprise a housing, the dispersive optics being connected to the housing and in particular arranged in the housing. For example, the housing may form the exit face and/or the entry face. The housing and the dispersive optics are made of the same material. In particular, the micro-optics can comprise further optical elements such as lenses, for example, which can also be made of the same material. By using the same material, the production of such micro-optics is simplified, so that the micro-optics can be produced from the corresponding material in one process step.

The micro-optics preferably comprise a housing, the dispersive optics being connected to the housing and in particular arranged in the housing. For example, the housing may form the exit face and/or the entry face. In this case, the housing and the dispersive optics are made of different materials. In particular, the material of the dispersive optics has a lower Abbe number in order to thus achieve a higher dispersion. Other optical elements of the micro-optics such as lenses, for example, can be made of the material of the housing, e.g. to reduce dispersion. As an alternative, these further optical elements can be made of the same material as the dispersive optics.

Preferably, the material of the dispersive optics has an Abbe number differing by at least 5, preferably by more than 10 and particularly preferred by more than 20 from the Abbe number of the material of which the housing is made. Here, as described above, the Abbe number of the material of the housing is higher than the Abbe number of the material of the dispersive optics.

Preferably, the splitting at the exit surface, in particular for visible light, in this case for the example wavelengths λ=450 nm and λ=650 nm, is between 1 μm and 50 μm, preferably between 2 μm and 30 μm and particularly preferred between 3 μm and 20 μm. Thereby, a sufficient splitting is achieved, so that different wavelengths of a pixel can be transmitted by different optical waveguides of the fiber of the fiber endoscope.

The micro-optics preferably comprises an aperture, the aperture being arranged in particular between the entry face and the dispersive optics. The imaging quality is further increased by the aperture.

Preferably, the aperture is arranged offset from the optical axis of the dispersive optics or from a fiber axis of the fiber endoscope, respectively.

Preferably, a cavity is provided between the entry face and the dispersive optics and/or between the dispersive optics and the exit face. By providing a cavity, corresponding refractive indices are achieved which are required to obtain the dispersive properties of the dispersive optics and the imaging properties of the further optical elements of the micro-optics, respectively. This can be achieved by the cavities which are filled with air or another gas, for example, and thus have a refractive index of approximately 1.

The present invention further relates to a method for manufacturing such micro-optics. The method comprises the following steps:
a) forming, by 3D laser writing, a housing from a first photoresist, the housing defining at least one cavity;
b) forming, in particular by 3D laser writing, dispersive optics in the at least one cavity.

Here, the housing and the dispersive optics can be formed at the same time by 3D laser writing. In particular, in 3D laser writing, the micro-optics are formed layer-by-layer and thus a simultaneous layer-by-layer forming of the housing and the dispersive optics can be performed. Alternatively, the housing is formed first and the dispersive optics are formed subsequently. Alternatively, the dispersive optics are formed first by 3D laser writing and the housing is formed thereafter.

Here, the dispersive optics can also be formed by 3D laser writing. Alternatively, the cavity defines the shape of the dispersive optics so that this cavity can be filled with photoresist and thereafter an exposure (in particular a UV flood exposure) can be performed to cure the photoresist.

Preferably, prior to forming the dispersive optics, a second photoresist is filled into the at least one cavity, the second photoresist and the first photoresist are different. In particular, the second photoresist has a higher dispersion and a lower Abbe number, respectively. Subsequently, the dispersive optics are formed from the second photoresist. Thus, the housing is used to receive the second photoresist from which the dispersive optics are then formed. Thus, the materials for the housing and the dispersive optics can be selected freely and can be introduced in two successive steps to form the housing and to subsequently form the dispersive optics from the different materials.

Preferably, an aperture is formed using an absorptive polymer. For this purpose, the housing comprises at least one, in particular annular recess. This recess can be open to the outside, for example, and can be partially or completely circumferential. In particular, the recess does not communicate with the at least one cavity of the housing. The absorptive polymer is then introduced into the recess and cured there. Curing can be performed, for example, by applying heat or light. As an alternative, curing is effected by a previously added initiator, so that the polymerization of the absorptive polymer is started.

Preferably, the micro-optics are formed directly on a fiber end face of a fiber of the fiber endoscope. Alternatively, the micro-optics are subsequently connected to the fiber end face, for example by gluing.

Preferably, micro-optics according to the above described micro-optics are formed by the method, so that features of the above description of the micro-optics can be freely combined with the method of the present invention.

Further, the present invention relates to an endoscope with a fiber. Here, the fiber is designed as an orderly optical waveguide bundle and thus comprises a plurality of optical waveguides. A first end, and in particular an end face of the first end of the fiber is connected to the micro-optics as described above. Further, a second end of the fiber is connected to imaging optics. The imaging optics comprising a dispersive element with a dispersion, so that the splitting of the light by the dispersive optics of the micro-optics is reversed. For this purpose, the dispersive element can comprise a dispersion opposite to the dispersive optics of the micro-optics. Here, the dispersive element of the imaging optics can have a dispersion of the same magnitude (but opposite or with a different sign) as the dispersive optics of the micro-optics, or a dispersion of a different magnitude. As an alternative, the dispersive element can have a substantially mirrored structure like the dispersive optics of the micro-optics. Due to the dispersive element of the imaging optics, the different color components or wavelength components of the respective pixels transmitted by the individual optical waveguides of the fiber are thus recombined into a single pixel.

Preferably, the dispersive element of the imaging optics can be changed to change the dispersion. Thus, the dispersion of the dispersive element can be adapted to the imaging optics. Here, in particular the position and/or the orientation of the dispersive element can be adapted.

Preferably, the dispersive element of the imaging optics is rotatable. Since in particular the micro-optics can have a non-rotationally symmetrical structure and, in particular, the splitting of the light occurs along an axis in the micro-optics, the orientation of the micro-optics must coincide with the orientation of the dispersive element of the imaging optics. This can be achieved by rotating the dispersive element in the imaging optics about its optical axis. This allows, for example, to change the fiber while retaining the imaging optics. Here, it is not necessary to connect the fiber to the imaging optics with the micro-optics in an intended orientation. Rather, a rotation of the dispersive element in the imaging optics can be used to achieve a correspondence between the orientation of the dispersive element and the micro-optics.

Preferably, the micro-optics has an outer diameter which substantially corresponds to the outer diameter of the fiber.

The fiber preferably has an outer diameter from 100 μm to 800 μm, preferably from 200 μm to 600 μm and particularly preferred from 300 μm to 500 μm.

Preferably, the optical waveguides of the fiber have a diameter of less than 3 μm, preferably less than 2 μm and particularly preferred less than 1.5 μm. The distance between individual optical waveguides in the optical waveguide bundle of the fiber is 2 μm to 5 μm,

### BRIEF DESCRIPTION OF THE DRAWINGS

The terms Fig., Figs., Figure, and Figures are used interchangeably in the specification to refer to the corresponding figures in the drawings

In the following, the invention is described in more detail by means of preferred embodiments with reference to the accompanying drawings.

In the Figures:

FIG. 1 illustrates a first embodiment of a fiber endoscope according to the present invention,

FIGS. 2A-2E show embodiments of the micro-optics according to the present invention,

FIGS. 3A-3C show further embodiments of the micro-optics according to the present invention,

FIGS. 4A, 4B show a schematic flow diagram of a method according to the present invention, and

FIGS. 5A, 5B show further embodiments of the micro-optics according to the present invention.

### DESCRIPTION OF THE INVENTION

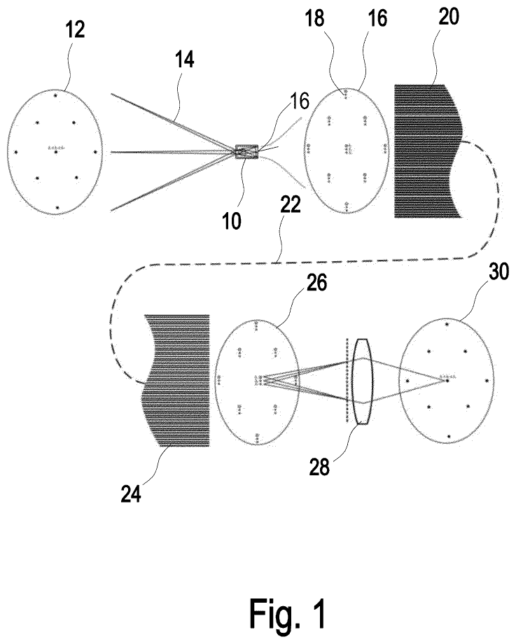

FIG. 1 schematically illustrates an endoscope according to the present invention with micro-optics 10. Exemplary pixels in an object plane 12 are imaged to an imaging plane 16 according to the beam structure 14, the imaging plane 16 coinciding with a fiber end face of a first fiber end 20 of a fiber 22 of the fiber endoscope. Here, it can be seen that individual pixels from the object plane 12 are split by the micro-optics 10 in dependence on the respective wavelength. This results in individual color components 18 of the respective pixels, which are then transmitted by the respective optical waveguide of the fiber 22 which is designed as an orderly optical waveguide bundle.

Imaging optics 28 are connected to a second fiber end 24 of the fiber 22. The individual color components 18 of the respective pixels are recombined into the individual pixels from an object plane 26, which coincides with the fiber end face of the second fiber end 24, via imaging optics 28 in an imaging plane 30. For this purpose, the imaging optics 28 have a dispersion, so that the individual color components are superimposed to form an individual pixel. Thus, the pixels in the image plane 30 again have almost complete color information. As an alternative to the imaging optics 28 illustrated in FIG. 1, further dispersive elements can be provided. It is further possible to image to infinity (i.e. without the focusing shown in FIG. 1), which can be imaged onto the user's eye by means of an eyepiece.

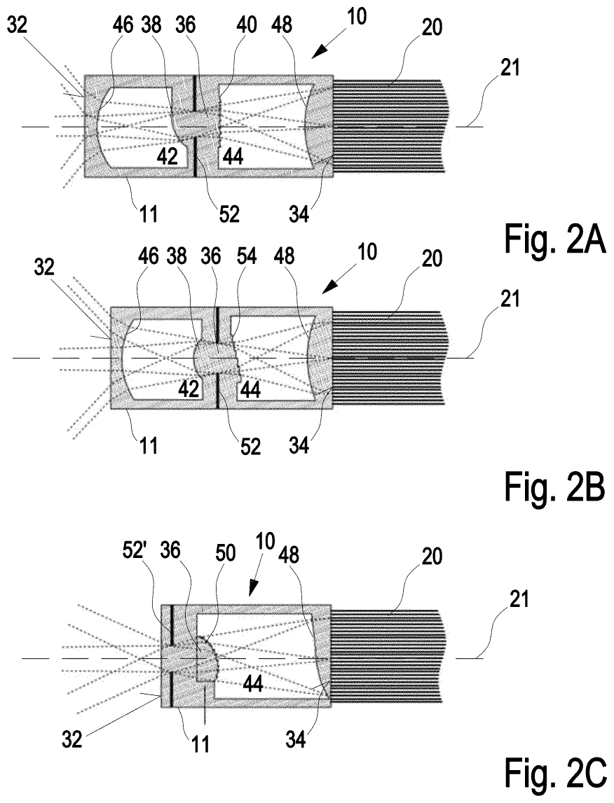

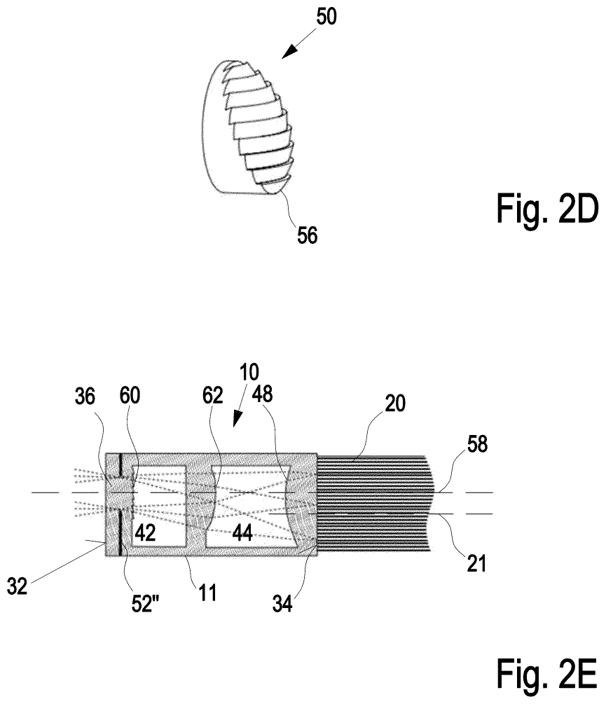

Reference is made hereinafter to FIG. 2A. FIG. 2A illustrates a first embodiment of the micro-optics 10. The first fiber end 20 of the fiber 22 is shown. A fiber axis 21 coincides with the axial direction of the micro-optics 10. The micro-optics 10 are directly and congruently connected to the fiber end face of the fiber 22. The micro-optics comprise a housing 11 with an entry face 32 and an exit face 34. The exit face 34 is directly connected to the fiber end face of the fiber 22. The entry face 32 and the exit face 34 are formed to be parallel, but can be inclined to each other in other embodiments. Further, the micro-optics 10 comprise a dispersive element 36 including a transmission grating 40. The exit face of the transmission grating 40 is parallel to the exit face 34 of the micro-optics 10. A cavity 42 is arranged between the dispersive element 36 and the entry face 32. Likewise, a further cavity 44 is formed between the dispersive optics 36 and the exit face 34 to spread the light rays and in particular to generate a desired refractive index difference. A first lens 46 is formed on the rear of the entry face 32. The surface of the dispersive optics 36, which is directed to the entry surface 32, is formed as a second lens 38. A third lens 48 is formed and directly connected to the exit face 34. It can be seen that at least the second lens 38 is non-rotationally symmetrical and, among other things, assumes a prismatic function which compensates for an image shift on the fiber end face by the dispersive element. Further, the micro-optics comprise an aperture 52 arranged between the entry face 32 and the transmission grating 40. This enhances the imaging properties.

Reference is made hereinafter to the further embodiments of the present invention. Here, identical or similar components are marked with identical reference numerals. Moreover, only the differences will be addressed hereinafter. Features of the individual embodiments can be freely combined with each other. This applies in particular to the arrangement and design of the dispersive optics 36, the provision of the cavities 42, 44, the design of the individual lenses 38, 46, 48 as well as their arrangement, the arrangement of the aperture and the like.

Reference is made to FIG. 2B. The dispersive optics 36 again comprises a transmission grating 54. However, the transmission grating 54 in FIG. 2B is inclined with respect to the exit face 34 or the entry face 32, respectively. Further, the transmission grating 54 is stair-shaped.

Reference is made hereinafter to FIGS. 2C and 2D. Here, the dispersive optics 36 have a transmission grating 50 which has segments 56 and a curved surface as shown in FIG. 2D. At the same time, the embodiment of FIG. 2C has no cavity, so that the dispersive optics 36 or the transmission grating 50 is directly connected to the entry face 32. Furthermore, the third lens 48 is designed as a freeform lens to compensate for anisotropies of the transmission grating 50. Here, the aperture 52′ is integrated into the entry face 32.

Reference is made hereinafter to FIG. 2E. Here, the dispersive optics 36 is designed as a transmission grating 60 and is directly connected to the entry face 32. A freeform lens 62 is arranged between the first cavity 42 and the second cavity 44. Here, the aperture 52″ has an axis 58 which is offset from the axial direction of the micro-optics 10 or the fiber axis 21, respectively.

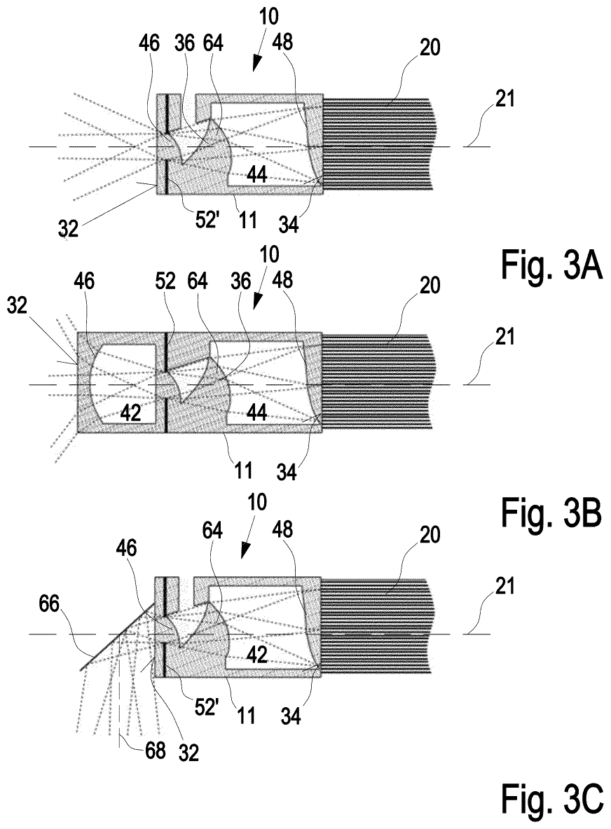

Reference is made hereinafter to FIG. 3A. Here, the dispersive optics 36 comprise a straight-view prism 64, wherein both the surface directed towards the exit face 34 and the surface directed towards the entry face 32 is curved and is in particular designed as a freeform surface. Here, the aperture 52′ is again integrated into the entry face 32. Furthermore, only one cavity 44 is provided. The third lens 48 can again be formed as a freeform lens, for example.

Reference is made hereinafter to FIG. 3B. Here, a first cavity 42 is formed between the entry face 32 and the dispersive optics 36 which comprises a straight-view prism 64, and a second cavity 44 is formed between the dispersive optics 64 and the exit face 34.

Reference is made hereinafter to FIG. 3C which corresponds to the embodiment in FIG. 3A and additionally comprises a deflection element 66, so that the optical axis 68 is deflected substantially rectangularly with respect to the fiber axis 21 or the axial direction of the micro-optics 10, respectively.

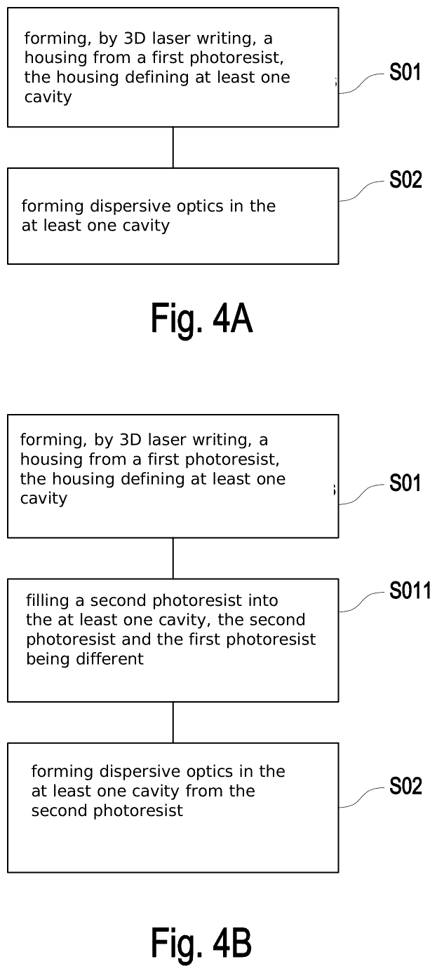

The method for producing the micro-optics 10 is schematically illustrated in FIG. 4A. The method comprises the following steps:

In step S01, forming, by 3D laser writing, a housing 11 from a first photoresist, the housing 11 defining at least one cavity.

In step S02, forming, in particular by 3D laser writing, dispersive optics 36 in the at least one cavity.

Here, the housing and the dispersive optics can be formed at the same time by 3D laser writing. In particular, in 3D laser writing, the micro-optics are formed layer-by-layer and thus a simultaneous layer-by-layer forming of the housing and the dispersive optics can be performed. Alternatively, the housing is formed first and the dispersive optics are formed subsequently. Alternatively, the dispersive optics are formed first by 3D laser writing and the housing is formed thereafter. Here, the dispersive optics 36 can also be formed by 3D laser writing. Alternatively, the cavity defines the shape of the dispersive optics so that this cavity can be filled with photoresist and thereafter an exposure (in particular a UV flood exposure) can be performed to cure the photoresist.

In another embodiment of the method, in particular when different materials are to be used for the housing and the dispersive optics, the method comprises the following steps:

In step S01, forming, by 3D laser writing, a housing 11 from a first photoresist, the housing 11 defining at least one cavity.

In step S011, filling a second photoresist into the at least one cavity, the second photoresist and the first photoresist being different.

In step S02, forming, in particular by 3D laser writing, dispersive optics 36 in the at least one cavity from the second photoresist.

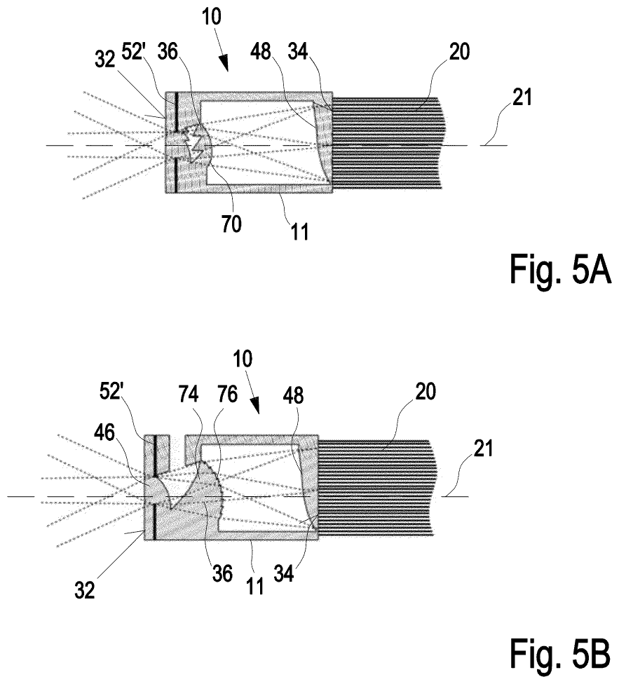

Reference is made hereinafter to FIG. 5A. Here, the dispersive optics 36 have a segmented prism 70. This allows for a compact design of the prism 70. The surface of the prism 70 can be inclined with respect to the exit face 34 and/or curved.

In contrast, FIG. 5B shows that the dispersive optics 36 comprises a transmission grating 76 on the surface facing the exit face 34, and that the opposite surface of the entry face 32 is formed as a prism 74 or the exit facet of the prism 74 is combined with the transmission grating 76.

Thus, a fiber endoscope and in particular micro-optics for a fiber endoscope is provided which can be easily miniaturized. This is possible because of the monolithic structure. It has been shown that it is possible to achieve a sufficient spectral/wavelength-dependent splitting of the individual pixels, even when using the same material for the housing and the dispersive optics. However, it is also possible to use different materials. Imaging errors can be corrected by the selected shape of the dispersive optics or of the further optical elements in the micro-optics. As such, the dispersive optics can be inclined with respect to the entry face and/or the exit face. As an alternative or in addition, the dispersive optics may comprise a curved surface. Thus, a substantially uniform splitting over the entire surface of the fiber end face is possible. By an appropriate selection of the shape of the dispersive optics, a linear relationship between the splitting and the wavelength is essentially achievable.

1. Micro-optics, in particular for connection to a fiber endoscope, comprising
an entry face and an exit face, the exit face being connectable to an end face of the fiber endoscope, and
dispersive optics arranged between the entry face and the exit face, so that light passing the dispersive optics is split at the exit face depending on the wavelength,
the micro-optics being monolithic.

2. The micro-optics according to claim 1, wherein the dispersive optics comprise a prism, in particular a straight-view prism.

3. The micro-optics according to claim 1, wherein the dispersive optics comprise a transmission grating.

4. The micro-optics according to claim 3, wherein the light exit face of the transmission grating is oriented parallel to the exit face and/or the transmission grating is stair-shaped.

5. The micro-optics according to claim 1, the dispersive optics are inclined with respect to the entry face and/or the exit face.

6. The micro-optics according to claim 1, wherein the dispersive optics have a first surface in the direction of the entry face and/or a second surface in the direction of the exit face, the first surface and/or the second surface being curved.

7. The micro-optics according to claim 1, wherein the dispersive optics have a first surface in the direction of the entry face and/or an opposite second surface in the direction of the exit face, the first surface and/or the second surface being non-rotationally symmetrical and in particular designed as a freeform surface.

8. The micro-optics according to claim 1, characterized in that the splitting of the light on the exit face takes place along an axis perpendicular to an axial direction of the micro-optics or the fiber endoscope.

9. The micro-optics according to claim 1, characterized by a housing, the dispersive optics being connected to the housing, the housing and the dispersive optics consisting of the same material.

10. The micro-optics according to claim 1, characterized by a housing, the dispersive optics being connected to the housing, the housing and the dispersive optics consisting of different materials.

11. The micro-optics according to claim 1, characterized by an aperture, the aperture being arranged in particular between the entry face and the dispersive optics.

12. The micro-optics according to claim 11, wherein the aperture is arranged offset from the optical axis of the dispersive optics or from a fiber axis of the fiber endoscope, respectively.

13. The micro-optics according to claim 1, wherein a cavity is provided between the entry face and the dispersive optics and/or between the dispersive optics and the exit face.

14. A method for manufacturing micro-optics, in particular according to claim 1, comprising the following steps:
a) forming, by 3D laser writing, a housing from a first photoresist, the housing defining at least one cavity;
b) forming dispersive optics in the at least one cavity.

15. The method according to claim 14, wherein the following steps are performed prior to forming the dispersive optics: filling a second photoresist into the at least one cavity, the second photoresist and the first photoresist being different; and
forming the dispersive optics from the second photoresist.

16. The method according to claim 14, wherein an aperture is formed from an absorptive liquid polymer, the housing comprising at least one in particular annular recess, the absorptive liquid polymer being introduced into the recesses and cured there.

17. The method according to claim 14, wherein the micro-optics is formed directly on a fiber end face of a fiber endoscope or is posteriorly connected to the fiber end face of the fiber endoscope.

18. An endoscope having a fiber with a first fiber end and a second fiber end, the fiber being formed as an optical waveguide bundle, a micro-optics according to claim 1 being connected to a first fiber end and in particular a fiber end face of the first fiber end and a second fiber end being connected to imaging optics, the imaging optics comprising a dispersive element with a dispersion, so that the splitting of the light is reversed by the dispersive optics of the micro-optics.

19. The endoscope according to claim 18, wherein the dispersive element of the imaging optics can be changed to change the dispersion, with in particular the position and/or the orientation of the dispersive element being changeable.
